(19) United States
(12) Patent Application Publication
(10) Pub. No.: US 2003/0014800 A1
(43) Pub. Date: Jan. 23, 2003

(54) Masks for eyeglasses

(76) Inventor: Judith P. Meyers, Greeley, CO (US)

Correspondence Address: GLENN L WEBB, P.O. BOX 951, CONIFER, CO, 80433

(21) Appl. No.: 09/910,415

(22) Filed: Jul. 20, 2001

(51) Int. Cl. A61F 9/00

(52) U.S. Cl. 2/15

(57) ABSTRACT

A mask for attachment to an eyeglass frame. A preferred embodiment of the present invention provides a mask formed from paper by paper-cutting. The mask includes two attachment members integrally formed with the mask. The attachment members then bend inward to secure over the eyeglass frame. The mask is also able to bend to allow it to be secured to differing sizes of eyeglass frames as well as shapes of individual heads.

### FIELD OF THE INVENTION

This invention relates to the field of masks.

### BACKGROUND OF THE INVENTION

Masks have played important roles throughout history in expressing cultural, religious, social and artistic beliefs. The use of masks dates back to the early Greeks for drama and festivals and possibly even earlier. Other early uses of masks included their use in Africa for tribal religious rites, in Egypt for sacrificial ceremonies, in Asia, for religious and social ceremonies, by Native Americans for honoring the dead and other traditional rites, and in many other indigenous cultures. Asian cultures used masks as early as 10,000 BC for ritual purposes. Masks, from these early times to the present, were used to represent symbolic forms, such as a deity, spirit, or a rite of passage, and for rituals, performances, celebrations and other events. They have been used by many cultures to depict beliefs and religious rites.

Presently, masks are used primarily as children's toys and for masked balls and celebrations, ranging from Halloween to Mardi Gras. Masks are also still used in many types of theatrical productions. Most masks traditionally were formed from wood, metal or ceramics. Modem masks tend to be manufactured from plastics for economy and comfort. Ornate full face paper-cut masks are used in Bejing Opera productions.

Most masks tend to be three-dimensional and are secured directly to the individual wearing the mask. Previously, masks have required some type of device for securing the mask to the individual wearing it, or held separately by the individual. These securing devices normally range from string, elastic cords, adhesives, or mount over the head or other portion of the body of the user. These masks are usually either relatively crude or expensive for more ornate or artistic masks. There are few if any masks presently that are elegant, ornate, relatively inexpensive and easily attachable for use by an individual.

One attempt at providing a novelty mask is disclosed in U.S. Pat. No. 3,009,163 and U.S. Design Pat. No. 184,703, both issued to Beauvais. The mask disclosed therein uses a three-dimensional molded disc-like member that is adhesively mounted to a spectacle-like frame.

Another spectacle-style attachment ornamentation is disclosed in U.S. Design Pat. No. 167,526, issued to Murphy. This mask uses a detachable ornamental clip that uses hooks to attach the clip onto an eyeglass frame. The detachable clip is intended to add ornamentation to the upper portion of the eyeglass frame to give it a different look.

### SUMMARY OF THE INVENTION

The present invention provides a mask formed from a substantially flat material and a process for using such a mask. The mask, in a preferred embodiment, is easily attachable to an eyeglass frame, and can be comfortably worn. The mask can be attached to almost any size of eyeglass frame.

In the preferred embodiment, the mask includes two attachment members that are formed integrally with the rest of the mask from a substantially flat material such as paper. In this preferred embodiment, the mask is formed by a paper-cutting process. In use, the two attachment members are bent inward slightly. This allows them to be attached inside of the earpieces or hinge portion of the eyeglass frame. Curved portions on the attachment members provide secure engagement to the frame.

The mask can be bent in order to accommodate differing sizes of frames and shapes of heads of the individuals using the mask. The masks can also include eyeholes, nosepieces, crests, and any other desired design elements. The masks can be of almost any size, shape or design.

While the preferred embodiment of the mask of the present invention are formed from paper by paper-cutting, other types of materials and forming processes can be used as well. For example, and without limitation, the masks can be formed from cardboard, plastic or any other substantially flat material.

These and other features of the present invention will be evident from the detailed description of preferred embodiments and from the drawings.

### BRIEF DESCRIPTION OF THE DRAWINGS

FIG. 1 is a front view of a mask of a preferred embodiment of the present invention.

FIG. 2 is an illustration of additional mask designs of the preferred embodiment of FIG. 1.

FIG. 3 is a side view of the embodiment of FIG. 1.

FIG. 4 is view of the mask of the embodiment of FIG. 1 attached to an eyeglass frame.

FIG. 5 is a side view showing the bending of the embodiment of FIG. 1.

### DETAILED DESCRIPTION OF PREFERRED EMBODIMENTS

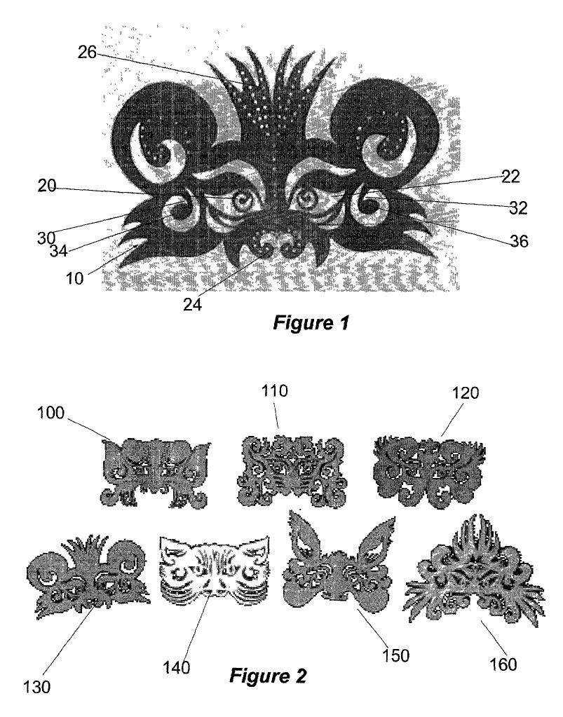

The present invention provides an elegant ornate mask that can be easily attached to eyeglass frames. A preferred embodiment of the present invention is illustrated in FIGS. 1-5. It is to be expressly understood that the exemplary embodiment discussed herein is provided for descriptive purposes only and is not meant to limit the scope of the present invention. The scope of the present invention is set forth in the appended claims, and encompasses the descriptive embodiments, the equivalents of the claimed elements disclosed herein as not only known by those skilled in the art but equivalents developed in the future.

As shown in FIG. 1, mask 10 includes an ornamental mask design. It is to be expressly understood that the present invention is not limited to the particulars of the mask design but includes any mask design that functions as claimed. For example, mask designs 100, 110, 120, 130, 140, 150, 160 are shown in FIG. 2. In the embodiment of FIG. 1, mask 10 is formed from a substantially flat material and/or a substantially pliable material. The preferred material is paper but other materials such as cardboard, plastic or other materials may be used as well.

Mask 10 includes eyeholes 20, 22, a nosepiece 24, and a crest 26. Mask 10 also includes attachment members 30, 32 discussed in greater detail below. All of these elements are formed from a single sheet of material. The shape of the elements of the mask can be of any desired design. Any of the above elements can be deleted except for the attachment members 30, 32 and other elements, such as a full face mask, can be added. Even the shape of the attachment members 30, 32 can be varied as desired. Other embodiments include shapes having unique ornamental designs, animal designs, famous individuals, ritualistic designs, or literally any desired shape. The designs on the masks can be formed, or easily printed directly onto the mask.

In the preferred embodiment illustrated in FIG. 1, the mask includes cut-out shapes around the eyeholes 20, 22 not only provide decorative features but enhance the sight and mobility of the mask wearer. Another interesting feature of the mask of the preferred embodiment of the present invention is the ability to wear the mask relatively close to the eyes. The closeness of the mask to the eyes of the mask wearer causes any minor obstructions or decorations on the mask to blur so not to substantially block the vision of the wearer.

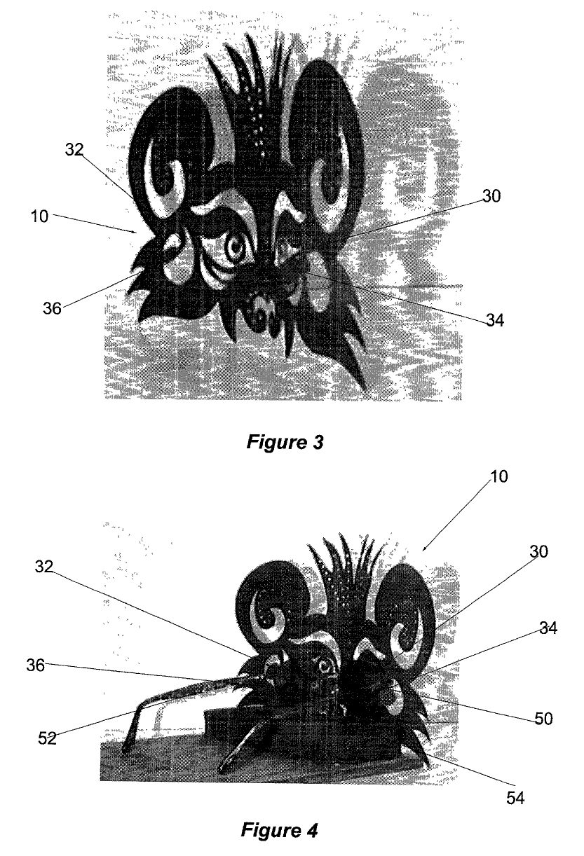

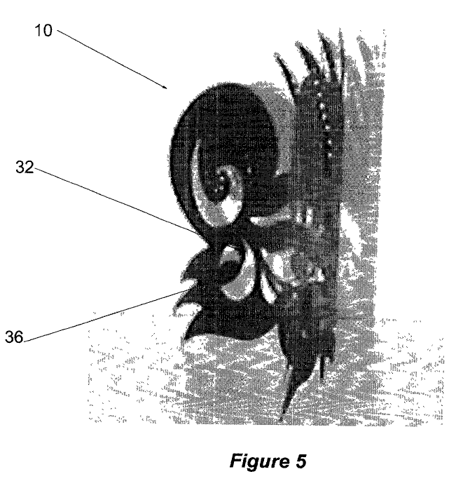

In use, the attachment members 30, 32 are bent slightly inward, as shown in FIG. 3. This allows the attachment members 30, 32 to be inserted inside the earpieces 50, 52 or hinges of an eyeglass frame 54 as shown in FIG. 4. The curved ends 34, 36 of the attachment members 30, 32 engage underneath the earpieces 50, 52 to secure the mask 10 to the frame 54. The mask also bends at the centerline of the mask, as shown in FIG. 5, to accommodate varying sizes of eyeglass frames as well as to fit more comfortably against the face of the individual wearing the eyeglass frame.

In the preferred embodiment, the attachment members 30, 32 are pliable so to be easily bent. In other embodiments, the attachment members may be more rigid or inflexible, with a living hinge or other type of hinge mechanism to enable the attachment members to bend inward for attachment to an eyeglass frame. Also, in other embodiments, the mask itself may be inflexible with either the attachment members being flexible or a hinge mechanism present to be allow the attachment members to bend inward for attachment to the eyeglass frame. In other embodiments, the attachment members are formed to be inward, or attachable onto the mask itself as separate members.

The preferred embodiment is formed by a paper-cut process to provide an ornate shape. It is also to be expressly understood that other processed can be used as well, such as die-cutting, molding and other types of forming processes. The intricate design of the mask 10, in a preferred embodiment, is paper-cut by laser, or even by hand with scissors or knives.

It is to be expressly understood that the above descriptive embodiment is proved strictly for explanatory purposes and is not meant to limit the scope of the claimed inventions.

What is claimed is:

1. A mask, wherein said mask comprises:
a mask formed from a substantially pliable material;
a first attachment member on one portion of said mask for attachment to a first portion of an eyeglass frame; and
a second attachment member on a second portion of said mask for attachment to a second portion of an eyeglass frame.

2. The mask of claim 1 wherein said substantially pliable material comprises:
paper.

3. The mask of claim 1 wherein said substantially pliable material comprises:
cardboard.

4. The mask of claim 1 wherein said substantially pliable material comprises:
plastic.

5. The mask of claim 1 wherein said substantially pliable material includes:
fabric.

6. The mask of claim 1 wherein said mask further comprises:
said mask formed from said substantially pliable material by paper-cutting.

7. The mask of claim 1 wherein said mask further comprises:
said mask formed from said substantially pliable material by die-cutting.

8. The mask of claim 1 wherein said mask further comprises:
said mask formed from said substantially pliable material by molding.

9. The mask of claim 1 wherein said mask further includes:
said first attachment member is bent inward from said substantially pliable material in order to be attached to the first portion of an eyeglass frame; and
said second attachment member is bent inward from said substantially pliable material in order to be attached to the second portion of an eyeglass frame.

10. The mask of claim 1 wherein said mask further includes:
said mask is bendable in order to accommodate differing widths of eyeglass frames.

11. The mask of claim 1 wherein said mask includes:
forming the entire structure of said mask from a single sheet of said substantially pliable material.

12. A mask, wherein said mask comprises:
a mask formed from a substantially flat material;
a first attachment member on one portion of said mask for attachment to a first portion of an eyeglass frame; and
a second attachment member on a second portion of said mask for attachment to a second portion of an eyeglass frame.

13. The mask of claim 12 wherein said substantially flat material comprises:
paper.

14. The mask of claim 12 wherein said substantially flat material comprises:
cardboard.

15. The mask of claim 12 wherein said substantially flat material comprises:
plastic.

16. The mask of claim 12 wherein said substantially flat material includes:
fabric.

17. The mask of claim 12 wherein said mask further comprises:
said mask formed from said substantially flat material by paper-cutting.

18. The mask of claim 12 wherein said mask further comprises:
said mask formed from said substantially flat material by die-cutting.

19. The mask of claim 12 wherein said mask further comprises:
said mask formed from said substantially flat material by molding.

20. The mask of claim 12 wherein said mask further includes:
said first attachment member is bent inward from said substantially flat material in order to be attached to the first portion of an eyeglass frame; and
said second attachment member is bent inward from said substantially flat material in order to be attached to the second portion of an eyeglass frame.

21. The mask of claim 12 wherein said mask further includes:
said mask is bendable in order to accommodate differing widths of eyeglass frames.

22. The mask of claim 12 wherein said mask includes:
forming the entire structure of said mask from a single sheet of said substantially flat material.

23. A mask, wherein said mask comprises:
a mask formed from a substantially pliable material;
a first attachment member integrally formed on one portion of said mask and bendable inward for attachment to a first portion of an eyeglass frame; and
a second attachment member integrally formed on a second portion of said mask and bendable inward for attachment to a second portion of an eyeglass frame.

24. The mask of claim 23 wherein said mask further includes:
said mask formed from a single sheet of said substantially pliable material.

25. The mask of claim 23 wherein said mask further includes:
said mask formed from a substantially flat material.

26. A process for using a mask having a first attachment member and a second attachment member, said process comprising:
attaching said first attachment member to a first portion of an eyeglass frame; and
attaching said second attachment member to a second portion of an eyeglass frame.

27. The process of claim 26 wherein said process further includes:
forming said mask from a substantially flat material.

28. The process of claim 26 wherein said process further includes:
forming said mask from a substantially flat material;
bending said first attachment member inward; and
bending said second attachment member inward.

29. The process of claim 26 wherein said process further includes:
forming said mask from a substantially pliable material.

30. The process of claim 26 wherein said process further includes:
bending said mask in order to accommodate differing widths of eyeglass frames.

31. The process of claim 26 wherein said process further includes:
forming said mask by paper-cutting.

32. The process of claim 26 wherein said process further includes:
forming said mask by die-cutting.

33. The process of claim 26 wherein said process further includes:
forming said mask by molding.

34. A mask wherein said mask comprises:
a face portion of said mask;
a first attachment member integrally formed with said face portion and bendable inward to attach to a first portion on an eyeglass frame; and
a second attachment member integrally formed with said face portion and bendable inward to attach to a second portion on an eyeglass frame.

35. The mask of claim 34 wherein said mask includes:
said mask is bendable to accommodate differing sizes of eyeglass frames.

36. The mask of claim 34 wherein said mask includes:
said mask formed by paper-cutting.

37. The mask of claim 34 wherein said mask includes:
said mask formed by die-cutting.

38. The mask of claim 34 wherein said mask includes:
said mask formed by molding.
